(19) United States
(12) Patent Application Publication
(10) Pub. No.: US 2025/0143913 A1
(43) Pub. Date: May 8, 2025

(54) INFLATABLE OSTOMY DEVICE

(71) Applicant: BOSTON SCIENTIFIC SCIMED, INC., Maple Grove, MN (US)

(72) Inventors: Serena SCOTT, Worcester, MA (US); Siavash Sam PARKHIDEH, Marlborough, MA (US); Tracy ANDREOTTI, Milford, MA (US); Jennifer LEWITZKY, Lexington, MA (US); Paul CARNE, Escanaba, MI (US); Daniel KEENAN, Charlton, MA (US); Brian LUIS, Boylston, MA (US)

(21) Appl. No.: 18/931,826

(22) Filed: Oct. 30, 2024

Related U.S. Application Data
(60) Provisional application No. 63/597,202, filed on Nov. 8, 2023.

(51) Int. Cl. A61F 5/448

(52) U.S. Cl. CPC A61F 5/448

(57) ABSTRACT

An ostomy device includes an inflatable member and a tubing. The inflatable member defines a hole extending therethrough. The inflatable member is configured to extend about a portion of an intestine proximate a stoma. In an operative configuration, the inflatable member movable between a deflated configuration, in which the stoma is maintained in an open position, and an inflated configuration, in which the inflatable member expands to decrease a size of the hole such and close the stoma. The tubing extends from a first end connected to the inflatable member to a second end. A channel of the tubing is in communication with an interior of the inflatable member such that inflation fluid is configured to flow through the tubing to move the inflatable member between the deflated and inflated configurations.

### PRIORITY CLAIM

The present disclosure claims priority to U.S. Provisional Patent Application Ser. No. 63/597,202 filed Nov. 8, 2023; the disclosure of which is incorporated herewith by reference.

### FIELD

The present disclosure relates to ostomy devices and, in particular, relates to ostomy devices including an inflatable member configured as an artificial sphincter for ostomy management.

### BACKGROUND

Ostomy procedures (e.g., colostomy, ileostomy) are performed for various patients with diseased or damaged bowels including, for example, colorectal cancer and diverticular disease. Current ostomy procedures include re-routing a part of the colon (or small intestine) to drain outside of the body by bringing an end of the colon to the abdominal wall, where edges of the colon are stitched to the skin to form an opening called a stoma. Stool is then able to drain from the stoma into a bag or pouch attached to the abdomen. Ostomy procedures, however, often result in post-operative complications and/or patient discomfort including, for example, leakage, odor, skin irritation from attachment of the ostomy bag, and social anxiety.

### SUMMARY

The present disclosure relates to an ostomy device which includes an inflatable member defining a hole extending therethrough, the inflatable member configured to extend about a portion of an intestine proximate a stoma, in an operative configuration, the inflatable member movable between a deflated configuration, in which the stoma is maintained in an open position, and an inflated configuration, in which the inflatable member expands to decrease a size of the hole such and close the stoma. The ostomy device also includes a tubing extending from a first end connected to the inflatable member to a second end, a channel of the tubing in communication with an interior of the inflatable member such that inflation fluid is configured to flow through the tubing to move the inflatable member between the deflated and inflated configurations.

In an embodiment, the hole of the inflatable member is configured to receive the portion of the intestine proximate the stoma therein so that, in the inflated configuration, the inflatable member expands to apply a radially inward pressure about the portion of the inflatable member received therein to close the stoma.

In an embodiment, the device further includes a bag configured to be attached to an abdominal wall about the stoma such that the tubing extends into an interior thereof.

In an embodiment, the device further includes a valve including a first port extending into the interior of the bag to be coupled to the second end of the tubing and a second port extending along an exterior of the bag to be coupled to an inflation device for providing inflation fluid to the inflatable member, the valve movable between an open configuration, in which a pathway between the first and second ports is open to allow a flow therebetween and inflate the inflatable member, and a closed configuration, in which the pathway between the first and second ports is closed.

In an embodiment, the second end of the tubing includes a luer connector which extends to an exterior of the bag and is configured to be connected to an inflation device.

In an embodiment, the device further includes a clamp configured to be clamped over the bag and the tubing to maintain the inflatable member toward the inflated configuration.

In an embodiment, the inflatable member forms a partial ring, extending from a first end to a second end.

In an embodiment, the inflatable member is configured be inserted through the stoma and received within the portion of the intestine proximate the stoma so that the hole of the inflatable member is aligned with a pathway through the intestine, the device further comprising a tube element extending from a connecting end connected to the hole of the inflatable member to an outlet end to direct intestinal contents passing through the hole of the inflatable member out of the stoma via the outlet end.

In an embodiment, the device further includes a secondary control element housed within the tube element and movable between a closed configuration, in which the secondary control element extends across a channel of the tube element to block flow of intestinal contents therethrough, and an open configuration, in which the secondary control element unlocks the channel of the tube element to permit a passage of intestinal contents therethrough.

In an embodiment, the secondary control element is configured as a balloon.

In an embodiment, the device further includes a faceplate coupled to the outlet end of the tube element; and an expandable spacer configured to extend between the faceplate and an exterior surface of an abdominal wall, in the operative configuration, a length of the spacer configured to be adjustable along a length of the tube element to attain a desired distance between the faceplate and the exterior surface of the abdominal wall.

In an embodiment, the faceplate includes a plurality of ports, each of which is configured to be connected to one or more of a plurality of fluid sources for controlling an inflation of the inflatable member, a movement of the secondary control element between the open and closed configurations, and adjustment of the length of the expandable spacer.

In addition, the present disclosure relates to a system for draining intestinal contents via a stoma. The system includes a plurality of inflatable members, each of which is configured to be positioned about a portion of an intestine, proximate an interior surface of an abdominal wall within which the stoma is formed, the inflatable members configured to be moved from a deflated configuration toward an inflated configuration, in which the inflatable members are expanded radially inward to squeeze a portion of the intestine received therein, the plurality of inflatable members configured to be moved from the deflated configuration toward the inflated configuration sequentially, from an inflatable member farthest from the stoma to an inflatable member closest to the stoma, so that intestinal contents are pushed through the intestine toward the stoma.

In an embodiment, the system further includes a tubing extending from a first end connected to the inflatable member farthest from the stoma to a second end, a channel of the tubing in communication with an interior of the inflatable member farthest from the stoma such that inflation fluid is configured to flow through the tubing to the inflatable member farthest from the stoma; and a channel connecting the plurality of inflatable members so that upon inflation of the inflatable member farthest from the stoma, inflation fluid is permitted to flow into a next adjacent inflatable member to sequentially inflate the plurality of inflatable members from the inflatable member farthest from the stoma to the inflatable member closest to the stoma.

In an embodiment, the system further includes an artificial sphincter having a substantially toroid shape to define a hole extending therethrough, the hole configured to receive a portion of the intestine proximate the stoma therethrough, in an operative configuration, the inflatable member movable between the deflated configuration, in which the stoma is maintained in an open position, and the inflated configuration, in which the inflatable member expands to decrease a size of the hole such that a radial inward pressure is applied to a portion of the intestine received therein to close the stoma.

In addition, the present disclosure relates to a method for performing an ostomy procedure. The method includes making an incision through an abdominal wall and drawing an end of a colon through the incision; drawing an end of the colon through the incision and positioning an inflatable member along an exterior surface of the abdominal wall and about a portion of the colon; stitching edges of the colon to the exterior surface of the abdominal wall to form a stoma such that the inflatable member is secured to between the exterior surface of the abdominal wall and the end of the colon; and moving the inflatable member between a deflated configuration and an inflated configuration to open and close the stoma, respectively, as desired.

In an embodiment, the moving the inflatable member between the deflated and inflated configurations includes providing inflation fluid to and from the inflatable member via a tubing extending therefrom.

In an embodiment, the method further includes attaching an ostomy bag to the exterior surface of the abdominal wall such that intestinal contents flow from the stoma into the ostomy bag when the stoma is open.

In an embodiment, the attaching the ostomy bag includes connecting a first port of a valve of the ostomy bag to the inflatable member and a second port of the valve to an inflation device for providing inflation fluid to the inflatable member, the valve movable between an open configuration, in which a flow between the inflatable member and the inflation device is permitted, and a closed configuration, in which flow between the inflatable member and the inflation device is prevented.

In an embodiment, the inflatable member is positioned along the exterior surface of a skin by implanting a base portion into an opening of the abdominal wall formed via the incision, the inflatable member housed within a head portion that is releasably coupled to the base portion.

### BRIEF DESCRIPTION

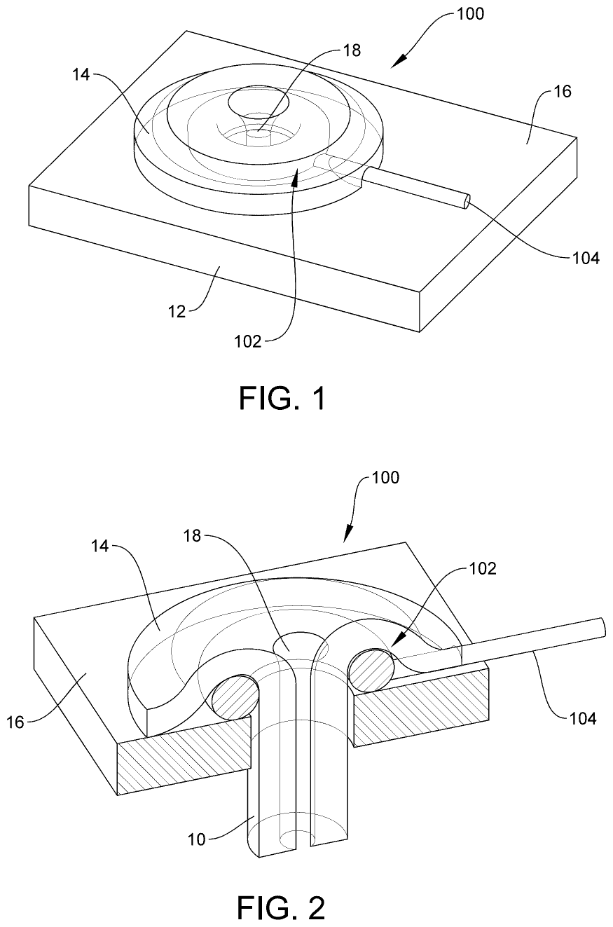

FIG. 1 shows a perspective view of an ostomy system according to an exemplary embodiment of the present disclosure.

FIG. 2 shows a cross-sectional perspective view of the ostomy system according to FIG. 1.

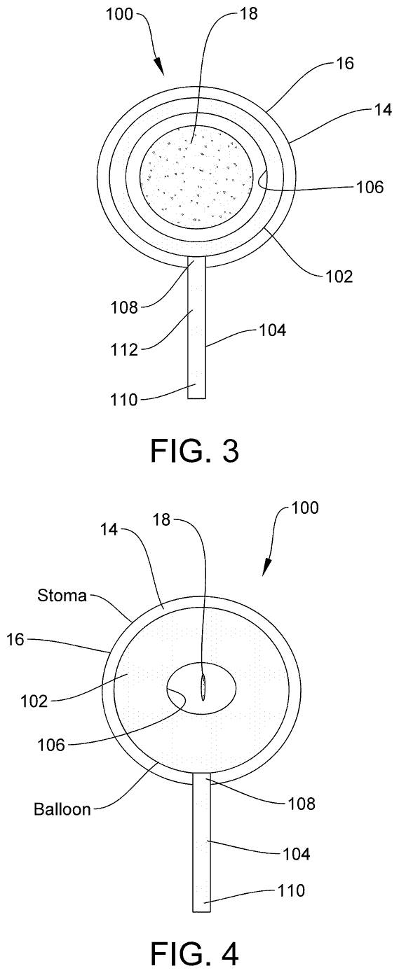

FIG. 3 shows a plan view of the ostomy system according to FIG. 1, in which an inflatable member thereof is in a deflated configuration to open a stoma.

FIG. 4 shows a plan view of the ostomy system according to FIG. 1, in which the inflatable member in an inflated configuration to close the stoma.

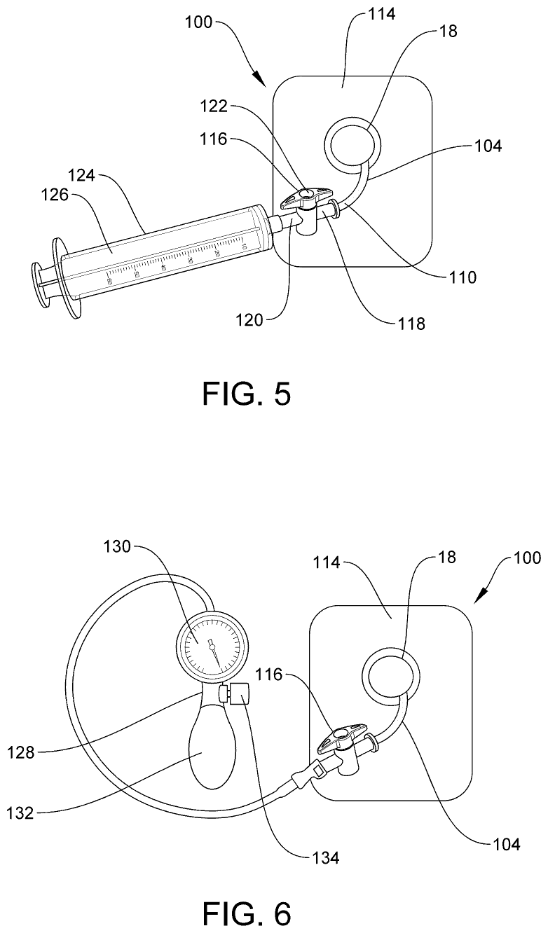

FIG. 5 shows a plan view of the ostomy system of FIG. 1, including a syringe.

FIG. 6 shows a plan view of the ostomy system of FIG. 1, including a hand pump.

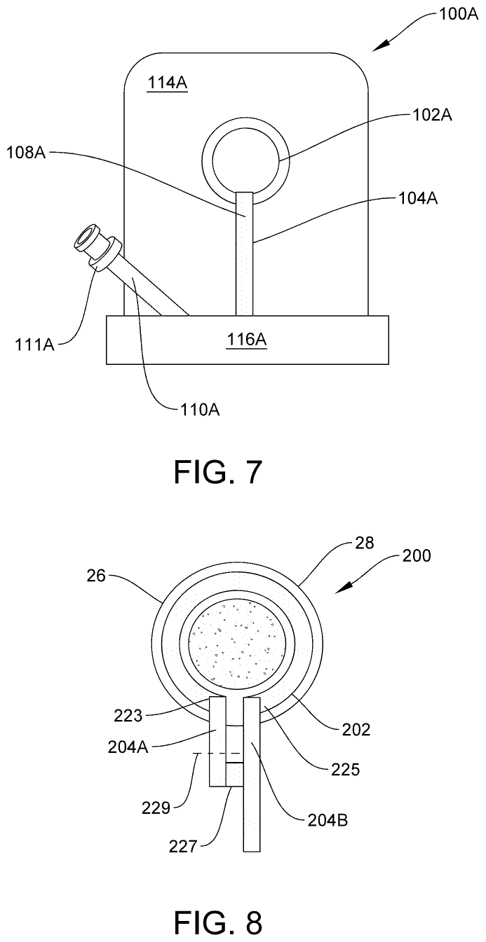

FIG. 7 shows a plan view of an ostomy system according to another exemplary embodiment, including a clamp.

FIG. 8 shows a plan view of an ostomy system according to yet another exemplary embodiment of the present disclosure.

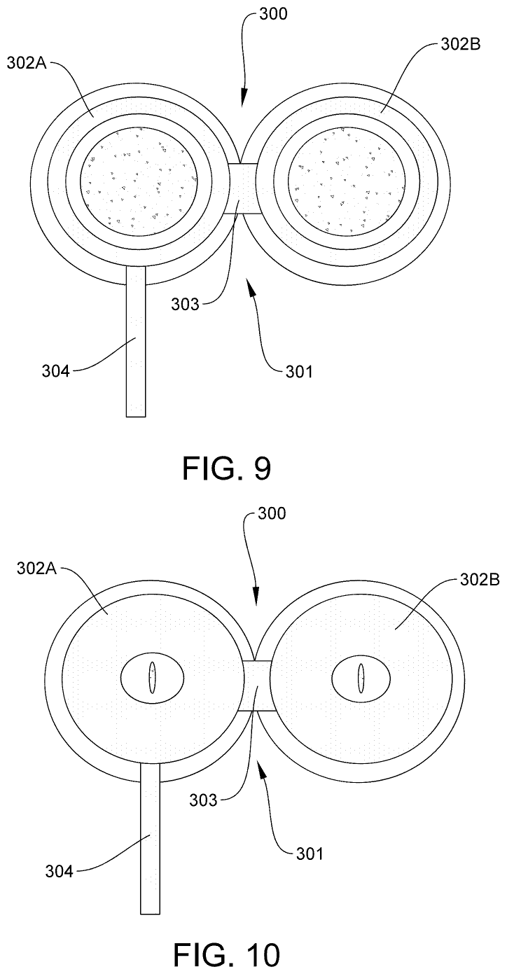

FIG. 9 shows a plan view of an ostomy device configured to be compatible with loop ostomy procedures according to another exemplary embodiment, with the ostomy device in a deflated configuration to open a stoma.

FIG. 10 shows a plan view of the ostomy device of FIG. 8, in an inflated configuration to close the stoma.

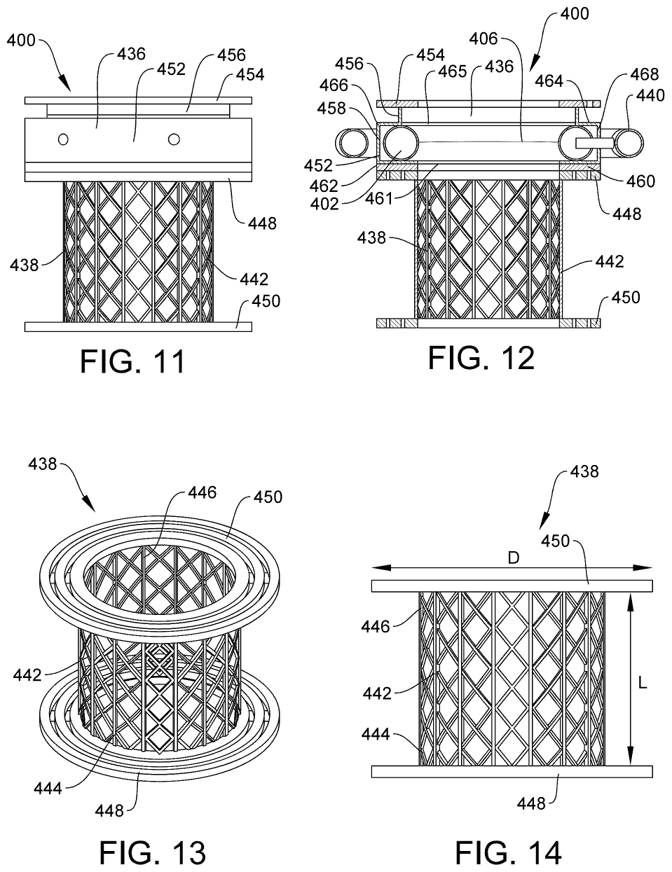

FIG. 11 shows a side view of an ostomy system comprising an ostomy device according to another exemplary embodiment of the present disclosure.

FIG. 12 shows a cross-sectional side view of the ostomy device according to FIG. 10.

FIG. 13 shows a perspective view of a base portion of the ostomy device according to FIG. 10.

FIG. 14 shows a side view of the base portion of the ostomy device according to FIG. 10.

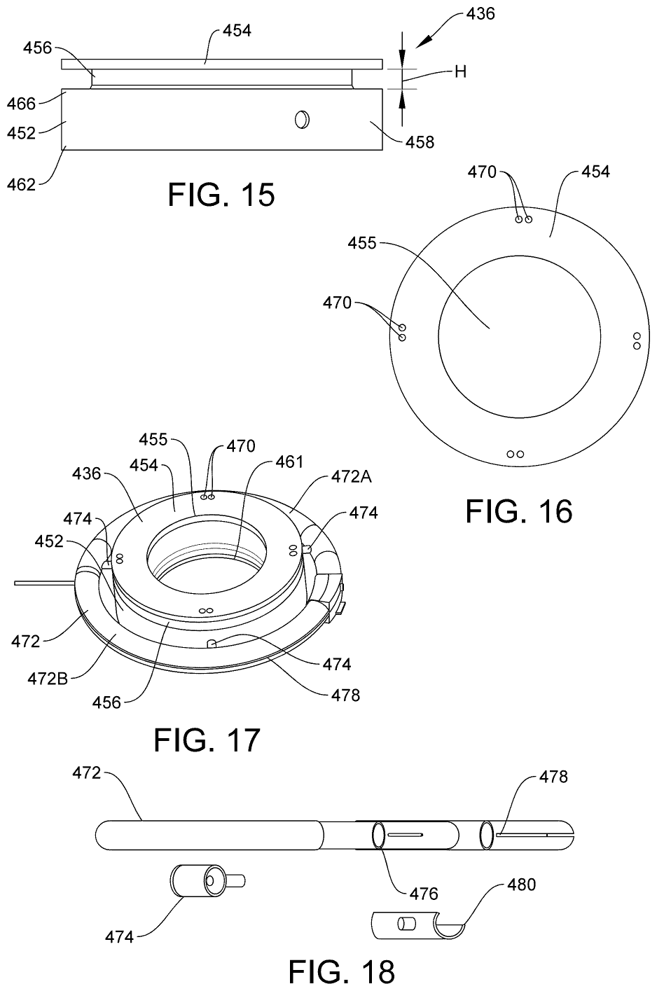

FIG. 15 shows a side view of a head portion of the ostomy device according to FIG. 10.

FIG. 16 shows a top plan view of the head portion of the ostomy device according to FIG. 10.

FIG. 17 shows a perspective view of the head portion and an irrigation system of the ostomy device according to FIG. 10.

FIG. 18 shows an exploded view of the irrigation system of the ostomy device according to FIG. 10.

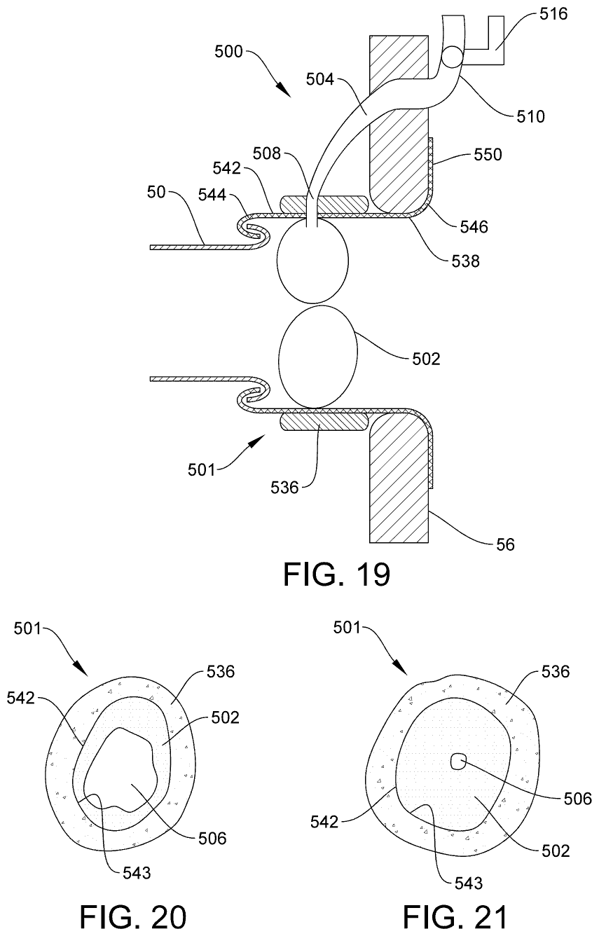

FIG. 19 shows a side view of an ostomy system according to another exemplary embodiment of the present disclosure.

FIG. 20 shows a cross-sectional view of an ostomy device according to the system of FIG. 18, in a deflated configuration in which a stoma is open.

FIG. 21 shows a cross-sectional view of the ostomy device according to FIG. 19, in an inflated configuration, in which the stoma is closed.

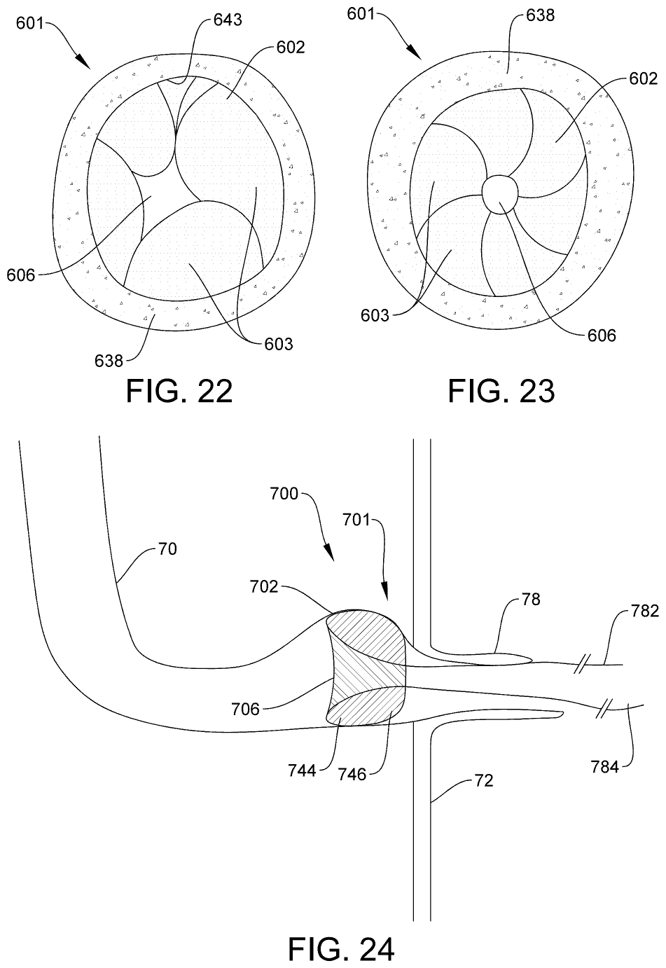

FIG. 22 shows a plan view of an ostomy device according to an alternate embodiment of the present disclosure in which an inflatable member is comprised of a plurality of lobes, the lobes in a deflated configuration so that a stoma is open.

FIG. 23 shows a plan view of the ostomy device according to the embodiment of FIG. 21, the lobes in an inflated configuration so that the stoma is closed.

FIG. 24 shows a cross-sectional side view of an ostomy system according to another exemplary embodiment of the present disclosure.

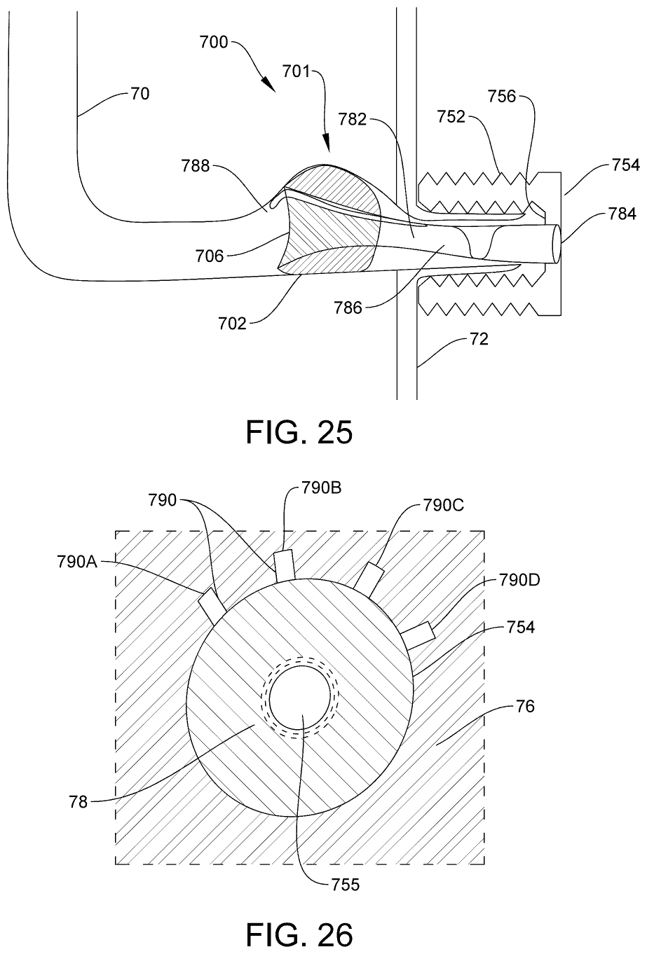

FIG. 25 shows a cross-sectional side view of the ostomy system of FIG. 23, according to a further exemplary embodiment.

FIG. 26 shows a plan view of a face plate of the ostomy system according to FIG. 24.

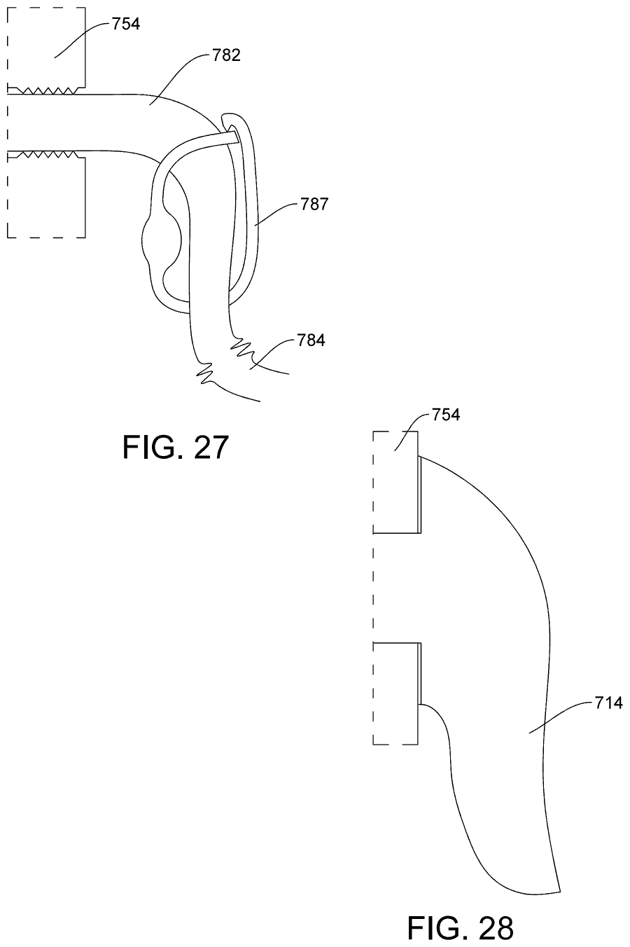

FIG. 27 shows a side view of the ostomy system of FIG. 24, according to another embodiment.

FIG. 28 shows a side view of the ostomy system of FIG. 24, according to yet another embodiment.

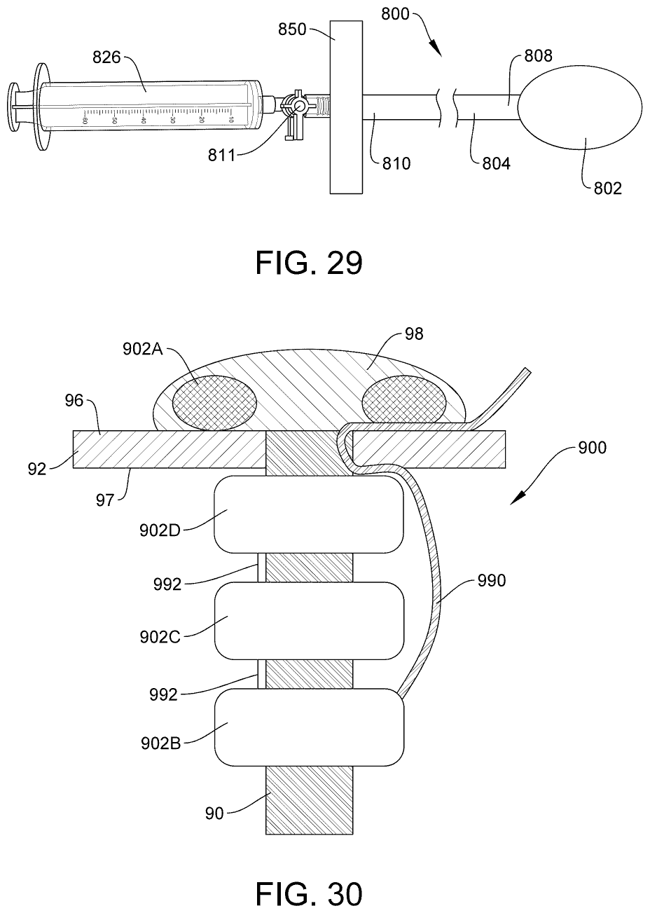

FIG. 29 shows a side view of an ostomy system according to another exemplary embodiment of the present disclosure.

FIG. 30 shows a cross-sectional side view of an ostomy system according to yet another exemplary embodiment of the present disclosure.

### DETAILED DESCRIPTION

The present disclosure may be further understood with reference to the following description and the appended drawings, wherein like elements are referred to with the same reference numerals. The present disclosure relates to an ostomy system and, in particular, relates to an ostomy device including an inflatable member that is configured to act as an artificial sphincter to control a flow of stool from a colon, through a stoma, and out of a patient body.

Exemplary embodiments of the present disclosure comprise an ostomy device including an inflatable member implantable proximate to a stoma (e.g., an opening in an abdominal wall that is connected to an end of an organ such as the small intestine or colon) to act as an artificial sphincter. In particular, the inflatable member is movable between a deflated configuration, in which the stoma is open to allow a passage of waste therethrough, and an inflated configuration, in which the stoma is closed to resist the passage of waste therethrough. Although the exemplary embodiments may specifically describe colostomy procedures in which the end of a colon is attached to an abdominal wall, it will be understood by those of skill in the art that the systems and methods of the present disclosure may be utilized for any ostomy procedure, including ileostomies, in which an end of the small intestine is re-routed to the abdominal wall.

As shown in FIGS. 1-4, an ostomy system 100 according to an exemplary embodiment of the present disclosure comprises an inflatable member 102 configured to be positioned about an intestine 10 (e.g., a colon) along an exterior of an abdominal wall 12, during surgical formation of a stoma 18. According to an exemplary embodiment, as shown in FIGS. 1-2, an end 14 of the intestine 10 is passed through an opening of the abdominal wall 12 and is secured (e.g., sutured) to the skin 16 of the abdominal wall 12, over the inflatable member 102, so that the inflatable member 102 is securely implanted between a bowel wall and an outer surface of the skin 16. The system 100 includes tubing 104 extending from the inflatable member 102 via which the inflatable member 102 may be moved between a deflated configuration (shown in FIG. 3), which opens the stoma, and an inflated configuration (shown in FIG. 4), which closes the stoma, to act as an artificial sphincter that controls a flow of waste (e.g., stool and gases) from the intestine.

In an exemplary embodiment, the inflatable member 102 has a substantially toroid shape, in which ends of a tubular element are connected to one another to form a substantially hollow circular ring permitting the inflatable member 102 to be positioned about the intestinal wall, proximate an end thereof, as discussed above. In particular, the inflatable member 102 of the exemplary embodiment is substantially ring or donut-shaped, defining a hole 106 extending therethrough. In an exemplary embodiment, the hole 106 is aligned with the opening through the abdominal wall to which the end of the intestine is connected. The inflatable member 102 may be formed of a variety of biocompatible polymers configured to enlarge and/or expand when filled with, for example, an inflation fluid (e.g., gas or liquid). For example, the inflatable member 102 may be formed of a silicone elastomer or any other suitable material as would be understood by those skilled in the art.

In the deflated configuration, as shown in FIG. 3, the hole 106 substantially corresponds to a cross-sectional area of the intestine, so that, when open the stoma allows stool to pass therethrough from the intestine. In the inflated configuration, as shown in FIG. 4, a diameter of the circular tubing which forms the donut-shape of the inflatable member 102 increases, thereby decreasing a diameter of the hole 106.

As the inflatable member 102 is moved toward the inflation configuration, the inflatable member 102 squeezes the intestinal wall around which it extends radially inward, closing the stoma and preventing waste from flowing therethrough from the intestine. Thus, the inflatable member 102 acts as an artificial sphincter to open and close the stoma, as desired. Although exemplary embodiments show and describe the tubular element forming the hollow tubular ring of the inflatable member 102 as having a substantially circular cross-section, the tubular element of the inflatable member 102 may have any of a variety of shapes so long as the inflatable member 102 is configured to be moved between the deflated and the inflated configurations to open and close the stoma, as desired.

The tubing 104 extends longitudinally from a first end 108 connected to the inflatable member 102 to a second end 110 connectable to a device for providing the inflation fluid to the inflatable member 102—e.g., a syringe. The tubing 104 includes a channel 112 extending therethrough, where the channel 112 is in communication with an interior of the inflatable member 102 so that the inflation fluid is provided to the interior of the inflatable member 102 via the tubing 104. In an operative configuration in which the inflatable member 102 is implanted between the bowel wall and the outer surface of the skin, the tubing 104 is extended from the stoma, along the exterior of the skin. In an exemplary embodiment, the tubing 104 may be flexible so that the tubing 104 may be connected to the syringe, or other inflation device, as desired.

The system 100 of this embodiment, as shown in FIG. 5, further comprises an ostomy bag 114 configured to be attached over the stoma (e.g., attached to the patient's skin), so that stool passing through the stoma is received and collected within the bag 114. In an exemplary embodiment, the bag 114 includes a valve 116 connected to the bag 114. In an exemplary embodiment, the valve 116 includes a knob 122 extending from an exterior of the bag 114, along with a first port 118 extending into an interior of the bag 114 and a second port 120 extending from the exterior of the bag 114.

The knob 122 is configured to open and close communication between the first and second ports 118, 120. In an exemplary embodiment, the first port 118 is configured to be connectable to the second end 110 of the tubing 104 via, for example, a luer connection. The second port 120 is configured to be connected to an inflation device 124 such as, for example, a syringe 126, as shown in FIG. 6. The second port 120 may be connected to the syringe 126 via, for example, a luer connection. Although the first and second ports 118, 120 are described as connectable to the tubing 104 and the syringe 126, respectively, it will be understood by those of skill in the art that the first and second ports 118. 120 may include any of a variety of connecting mechanisms including, for example, a friction fit. In an exemplary embodiment, the valve 116 may be permanently connected to the bag 114, but may be easily connected and disconnected from the inflation device 124 and the tubing 104.

In an exemplary embodiment, the valve 116 is movable between an open configuration when the inflation fluid is provided to the inflatable member 102 via the syringe 126 to the inflatable member, and a closed configuration in which the fluid is prevented from passing between the syringe 126 and the tubing 104. To move the inflatable member 102 from the deflated configuration toward the inflated configuration, the knob 122 is rotated to move the valve 116 from the closed configuration toward the open configuration. The syringe 126, which is connected to the second port 120, may then be depressed to inflate the inflatable member 102.

Once the inflatable member 102 has been inflated, as desired, the valve 116 may be moved from the open configuration to the closed configuration to trap the inflation fluid (e.g., air or water) therein. It will be understood by those of skill in the art that the syringe 126 is configured to provide and remove the inflation fluid to/from the inflatable member 102, as desired. For example, the syringe 126 may provide the inflation fluid to the inflatable member 102 to inflate the inflatable member 102 and close the stoma. When it is desired to open the stoma, valve 116 is moved to the open configuration and the syringe is operated to remove the inflation fluid from the inflatable member 102 to move the inflatable member 102 from the inflated configuration to the deflated configuration.

In an exemplary embodiment, the syringe 126 is a locking syringe including locking mechanisms configured to lock the syringe 126 at any of a variety of positions to hold the system 100 in a desired pressurized configuration, until the valve 116 is moved to the closed configuration. In an alternative embodiment, the system 100 includes a check valve inserted between the tubing 104 and the first port 118 at, for example, the luer connection at the second end 110 of the tubing 104. As would be understood by those of skill in the art, the check valve would aid in controlling sudden pressure changes when connecting and/or disconnecting the syringe 126 from the second port 120. The check valve would also reduce and/or prevent any unintended backflow from internal body pressures, which could unintentionally deflate the inflatable member 102 during normal use.

In another embodiment, as shown in FIG. 6, the inflation device 124 includes a hand pump 128 including a pressure gauge 130. The hand pump 128 may be connected to the valve 116 of the bag 114 in a manner substantially similar to the connection of the syringe 126 described above. In an exemplary embodiment, the hand pump 128 includes a bulb 132 configured to be squeezed to provide fluid to the inflatable member 102 to move the inflatable member 102 from the deflated configuration to the inflated configuration, and a knob 134 configured to be operated to vent air when moving the inflatable member 102 from the inflated configuration toward the deflated configuration.

In addition, the pressure gauge 130 may be set to vent automatically at a threshold pressure (e.g., a maximum pressure deemed safe for the stoma or any component of the system 100, or pressure set by the user). The pressure gauge may include markings indicating target inflation pressures for achieving a tight seal of the stoma without overinflating the inflatable member 102. According to an exemplary embodiment, in some cases, the patient may open the valve 116 to open the stoma when it is desired to empty the intestinal contents (i.e., waste). This may be done a few minutes before the patient is ready to empty the bag 114 so that the stool has sufficient time to flow into the bag 114.

Although the exemplary embodiment of the system 100 is shown as including the bag 114, it will be understood by those of skill in the art that the system 100 described above may, if desired, be utilized alone, without the bag 114. When used alone, the tubing 104 may be connected directly to the inflation device 124 (e.g., syringe, hand pump) to deflate/inflate the inflatable member 102, as desired. In an exemplary embodiment, the inflatable member 102 may be maintained in the inflated configuration, closing the stoma until the patient is ready to use the bathroom.

The inflatable member 102 may then be moved toward the deflated configuration to open the stoma so that the stool may be drained directly from the stoma into the toilet or other disposal system. The bag 114 may be used as a backup to reduce the chances of leakage. It will also be understood by those of skill in the art that although the system 100 is specifically described with respect to a syringe 126 and/or a hand pump 128, the inflation device 124 connectable to the tubing 104 to provide the inflation fluid to/from the inflatable member 102 may include any of a variety of devices capable of inflating and deflating the inflatable member 102, as desired.

According to an exemplary method utilizing the system 100, a portion of a target organ (e.g., the colon or ileum) is passed through an incision in an abdominal wall. The inflatable member 102 is positioned about this portion of the target organ (e.g., the colon), along an exterior surface of a skin of the abdominal wall. Edges along the end of the portion of the target organ may then be sutured to the skin to secure the inflation member 102 between a wall of the target organ and the skin to form a stoma through which waste may be passed. Edges of the target organ may be stitched to the skin so that the tubing 104 extends out of the stoma so that it is accessible to a user (e.g., patient) of the system 100. As described above, the system 100 may be utilized alone, or in combination with the bag 114 as backup for preventing leakage.

Where the system 100 is utilized without the bag 114, the tubing 104 may be connected to an inflation device such as, for example, a syringe or hand pump. The valve 116 would be directly connected to the tubing 104 to keep the system inflated. The syringe may, for example, be connected to the tubing 104 or the valve 116 via a luer connection. As described above, the inflation device may be used to move the inflatable member 102 between the deflated configuration and the inflated configuration to open and close the stoma, respectively. The inflation device provides fluid (e.g., air or water) to the inflatable member 102 to cause the inflatable member 102 to expand, decreasing a size of the hole 106 defined thereby.

As the inflatable member 102 is moved from the deflated configuration to the inflated configuration, the expanding inflatable member 102 presses a portion of the target organ, about which it extends, radially inward to close the stoma. The inflatable member 102 is moved from the inflated configuration to the deflated configuration by drawing the air out of the inflatable member 102, releasing the radially inward pressure about the end of the target organ to allow the stoma to open.

The bag 114 may be positioned over the stoma in a manner substantially similar to conventional ostomy bags. As described above, however, the bag 114 includes the valve 116. In an exemplary embodiment, the first port 118 of the valve 116, which extends along an interior of the bag 114, may be connected to the second end 110 of the tubing 104 while the second port 120 of the valve 116, which extends from the exterior of the bag 114, is connected to the inflation device 124 (e.g., the syringe 126 or the hand pump 128). The knob 122 of the valve 116 may be used to open the valve 116, allowing fluid to pass between the inflation device 124 and the inflatable member 102.

While the valve 116 is in the open position, the inflation device 124 is used to supply fluid to the inflatable member 102 to move the inflatable member 102 to the inflated configuration. The inflatable member 102 may be maintained in the inflated configuration, closing the stoma, by closing the valve 116. When it is desired to allow waste to pass through the stoma, the valve 116 is moved to the open position, and the inflation device 124 used to remove the fluid from the inflatable member 102 opening the stoma to allow stool to flow therethrough for collection within the bag 114. It will be understood by those of skill in the art that, if so desired, the inflation device 124 may be disconnected from the valve 116 while the valve 116 is in the closed position.

When the user does not wish to use the bag 114, the stoma may be maintained in the closed position (the inflatable member 102 in inflated configuration) until the patient has access to a bathroom and/or when it is desired to empty waste from the target organ. The inflatable member 102 may then be moved to the deflated configuration to open the stoma. The stool may then be released directly into, for example, a toilet or other container.

A system 100A according to another exemplary embodiment, as shown in FIG. 7, is substantially similar to the system 100 except as noted below. The system 100A comprises an inflatable member 102A configured to be moved between a deflated configuration and an inflated configuration to open and close a stoma, respectively, via tubing 104A configured to be connected to an inflation device (e.g., syringe). Similarly to the system 100, the system 100A may further comprise an ostomy bag 114A. However, the bag 114A does not include a valve. While a first end 108A of the tubing 104A is contained within the bag 114A when the bag 114A is in an operative position over the stoma. A second end 110A of the tubing 104A is configured to extend to an exterior of the bag 114A so that a luer connection 111A thereof may be connected to the inflation device. A clamp 116A may be used to both clamp the bag 114A and the tubing 104A closed, when the inflatable member 102A is in the inflated configuration, to hold the stoma closed until it is desired to empty the colon of its contents.

A system 200 according to yet another exemplary embodiment, as shown in FIG. 8, is substantially similar to the systems 100, 100A described above, except as noted below. Similarly to the system 100, the system 200 comprises an inflatable member 202 that is configured to be positioned about an intestine, along an exterior surface (e.g., skin 26) of an abdominal wall, so that moving the inflatable member 202 between deflated and inflated configurations opens and closes a stoma 28, respectively. Rather than forming a full ring as described with respect to the inflatable member 102, however, the inflatable member 202 forms an incomplete and/or partial ring extending from a first end 223 to a second end 225. In other words, the inflatable member 202 may be substantially C-shaped, sized to extend about a substantial portion of a periphery of the intestine. Rather than a single tube, the system 200 includes a pair of tubes 204A, 204B, cach of which may extend from a corresponding one of the first and second ends 223, 225 to an exterior of the stoma 28 so that inflation fluid (e.g., gas or liquid) may be provided to the inflatable member 202 via one or more of the tubes 204A, 204B. In an embodiment, the pair of tubes 204A, 204B may be connected to one another via a connector 227, which also extends exterior to the stoma 28. It will be understood by those of skill in the art that the system 200 allows for a simple, non-invasive removal thereof, if necessary. In particular, one or both of the tubes 204A, 204B may be cut along a cut line 229 so that the inflatable member 202 may be pulled or drawn out of the stoma 28.

A system 300 according to yet another exemplary embodiment, as shown in FIGS. 9-10, is substantially similar to the systems 100, 200 described above except as noted below. Rather than a single inflatable member as included in the systems 100 and 200, the system 300 comprises an ostomy device 301 including a pair of inflatable members 302A, 302B so that the system 300 is compatible with loop ostomies in which a loop of the colon (or other organ) is pulled through an incision formed in the abdominal wall. The loop is opened and edges thereof are stitched to the skin to form a stoma with two openings. Each of the inflatable members 302A, 302B is substantially similar to the inflatable member 102, as described above, and may be implanted over the skin, secured in place about a corresponding opening of the stoma when the edges of the target organ are stitched to the skin.

The inflatable members 302A, 302B may be connected to one another via a connecting portion 303 such that both of the inflatable members 302A, 302B may be deflated (see FIG. 9) and inflated (see FIG. 10) via a single tubing element 304. However, in an alternative embodiment the inflatable members 302A and 302B may be separate from one another and served by separate tubing elements as would be understood by those skilled in the art. It will also be understood by those of ordinary skill in the art that, similarly to the systems described above, the system 300 may be utilized with an ostomy bag, if so desired, in a manner substantially similar to the systems 100 described above.

As shown in FIGS. 11-17, a system 400 according to another exemplary embodiment is substantially similar to the system 100 described above except as noted below. The system 400 comprises an inflatable member 402 (e.g., a toroidal member) configured to open and close a stoma, as desired. As shown in FIGS. 11-12, the inflatable member 402, however, is housed within a head portion 436, which is releasably coupled to a base portion 438 configured to be implanted within an opening in the abdominal wall about an end of a portion of the target organ. When the system 400 is in an operative configuration in which the base portion is implanted within the abdominal opening, the head portion 436 extends along an exterior surface of the skin.

In this embodiment, the inflatable member 402 is housed within the head portion 436, which, in an operative configuration, extends along an exterior of the abdominal wall about a portion of the target organ that extends out of the base portion 438. Thus, when edges of an end of the target organ are stitched to the skin to form a stoma, the inflatable member 402, within the head portion 436, is secured between the skin and an exterior of the target organ. The inflatable member 402 may be moved between a deflated and inflated configuration to open and close the stoma, respectively, to empty the bowels, as desired. In an exemplary embodiment, the head portion 436 is connected to an injection system 440 (e.g., an irrigation system) in communication with the inflatable member 402 to move the inflatable member 402 between the deflated and inflated configurations.

It will be understood by hose of skill in the art that the base portion 438 may be implanted within the abdominal wall to provide support to the target organ and the abdominal wall, thereby reducing risk of a hernia. The head portion 436 remains exterior to the abdominal wall so that the head portion 436 may be decoupled from the base portion 438 to replace the head portion 436 and/or inflatable member 402 in the case of, for example, an injection system failure, leaks a burst inflatable member 402, or other failure sure to use of the inflatable member 402 over an extended period of time.

As shown in FIGS. 13-14, the base portion 438 includes a tubular body 442 extending from a first end 444 to a second end 446. The tubular body 442 of this embodiment is formed of a biocompatible material (e.g., titanium) having a mesh configuration. The first end 444 includes a first flange 448 while the second end 446 includes the second flange 450. A length L of the tubular body 442 is configured to correspond to a length of the opening, which extends through the abdominal wall from an interior surface to an exterior surface (e.g., skin) thereof.

In other words, the length L of the tubular body 442 corresponds to a width of the abdominal wall—i.e., a distance between the interior surface of the abdominal wall and the exterior surface of the abdominal wall. The first flange 448 engages the interior surface of the abdominal wall while the second flange 450 engages the exterior surface of the abdominal wall, securing the tubular body 442 of the base portion 438 within the opening. A diameter D of the one of the first and second flanges 448, 450 of this embodiment corresponds to a desired size of the stoma which depends on, for example, the anatomy involved in the ostomy procedure. For example, a diameter of the tubular body 442 may substantially correspond to a diameter of a target portion of the intestine (e.g., colon, ileum) of the ostomy procedure.

In an exemplary embodiment, the first and second flanges 448, 450 have the same size and shape. It will be understood by those of skill in the art, however, that each of the first and second flanges 448, 450 may have any of a variety of sizes and shapes so long as the first and second flanges 448, 450 are sized and shaped to engage the interior and exterior surfaces, respectively, of the abdominal wall to secure the tubular body 442 within the opening extending therethrough.

As shown in FIG. 15, the head portion 436 includes a housing 452 configured to house the inflatable member 402 and a plate 454 connected to the housing 452 via a reduced diameter portion to form a stepped groove 456 about an exterior of the head portion 436. The housing 452 is formed via an outer wall 458, a first surface 460 at a first end 462 of the outer wall 458 and a second surface 464 at a second end 466 of the outer wall 458. The first surface 460 may include features for facilitating a releasable coupling with the base portion 438.

For example, the first surface 460 and the second flange 450 of the base portion 438 of this embodiment include corresponding engaging features. The outer wall 458, the first surface 460 and the second surface 464 define a cavity 468 within which the inflatable member 402 is housed. In an exemplary embodiment, the first and second surfaces 460, 464 substantially correspond in size and shape to the second flange 450, including holes 461, 465 extending therethrough, so that a portion of the target organ (e.g., colon, ileum, etc.) that is passed through the tubular body 442 is also able to pass through the holes 461, 465 of the first and second surfaces 460, 464 of the housing 452 and a hole 406 of the inflatable member 402 housed therein.

The plate 454, as shown in FIG. 16, includes a hole 455 extending therethrough in alignment with the holes 461, 465 of the housing 452 to permit passage of a portion of the target organ therethrough. The plate 454 of an exemplary embodiment further includes a plurality of suture holes 470 along a periphery thereof configured to permit suturing of the end of the target organ and/or skin thereto. It will be understood by those of skill in the art that the stepped groove 456, which is immediately adjacent the plate 454, is configured to permit manipulation of the head portion 436 via, for example, the surgeon, and/or placement of an ostomy bag (not shown) or other accessory which may be used in conjunction with an ostomy procedure. A height H of the stepped groove—e.g., a distance between the plate 454 and the second surface 464—may be selected to, for example, facilitate connection with an ostomy bag or other accessory.

As shown in FIGS. 17-18, the injection system 440 may function substantially similarly to a syringe and includes a hollow tube 472 (e.g., having a circular shape) connected to the head portion 436, in communication with the inflatable member 402 housed therein, via a plurality of hollow legs 474 that are welded to the housing 452. A first portion 472A of the hollow tube 472 is filled with a liquid (e.g., water) which may be pressurized into the inflatable member 402, while a second portion 472B of the hollow circular tube contains a plunger 476 which may be slid through the hollow tube 472 to pressurize the inflatable member 402.

In an exemplary embodiment, the legs 474 connecting the first portion 472A of the hollow tube 472 to the head portion 436 are also sealed within the inflatable member 402 so that, as the plunger 476 is moved from an open position toward a closed position, the plunger 476 pushes the fluid through the legs 474 to pressurize the inflatable member 402. The second portion 472B may include a rail 478 along which a handle 480 configured for moving the plunger 476 between the open and the closed positions may be slid.

Similarly to the inflatable member 102, the inflatable member 402 expands as it inflates, decreasing a size of the hole 406 to push the portion of the target organ received therein radially inward to close the stoma. When it is desired to open the stoma, the plunger 476 is moved toward the closed position, which depressurizes the inflatable member 402, releasing the radial inward pressure on the target organ and allowing the stoma to open.

The head portion 436 and/or the injection system 440 may be formed of any suitable material such as a metal (e.g., stainless steel) configured to support the pressures anticipated and to increase shelf life. Similarly to the system 100, the inflatable member 402 may be formed of an elastomeric material such as, for example, silicone elastomer.

According to an exemplary method utilizing the system 400 for an ostomy procedure, an incision is made in an abdominal wall to form an opening between the interior and exterior surfaces of the abdominal wall. The base portion 438 is positioned in the opening of the abdominal wall such that the first flange 448 engages the interior surface of the abdominal wall and the second flange 450 engages the exterior surface (i.e., skin) of the abdominal wall such that the base portion 438 is secured within the opening and the head portion 436 extends along the exterior of the skin. The end of a target organ (e.g., the colon) is then drawn through the tubular body 442 of the base portion 438, through the head portion 436 and the hole 406 of the inflatable member 402 housed therewithin. The edges of the colon and the skin are sutured to the suture holes 470 of the plate 454 of the head portion 436 to form a stoma.

The inflatable member 402 may be moved from the deflated configuration toward the inflated configuration to close the stoma until it is desired to empty the bowel. To inflate the inflatable member 402, the handle 480 of the injection system 440 is slid along the hollow tube 472 from the open position to the closed configuration to move the plunger 476 therewithin in a first direction. Movement of the plunger 476 within the hollow tube 472 causes the fluid within the first portion 472A to pressurize the inflatable member 402, causing it to expand and apply radially inward pressure on the portion of the colon about which it extends so that the stoma is closed.

When it is desired to empty the bowel, the handle 480 is moved from the closed position

to the open position sliding the plunger within the hollow tube 472 in a second direction, opposite the first direction, to depressurize the inflatable member 402. As the inflatable member 402 deflates, the radially inward pressure on the colon is released, thereby opening the stoma. As will be understood by those of skill in the art, the system 400 may be used with or without an ostomy bag. When utilized with an ostomy bag, the bag is coupled to the head portion 436 by, for example, engaging a portion of the bag with the stepped groove 456, so that the stool from the bowel is collected in the bag. It will be understood by those of skill in the art that the inflatable member 402 may be moved between the deflated and inflated configurations via the injection system 440 to open and close the stoma, as desired.

As shown in FIGS. 19-21, a system 500 according to another exemplary embodiment of the present disclosure is substantially similar to the system 400 described above except as noted below. The system 500 comprises an ostomy device 501 including a base portion 538 configured to be implanted within an opening extending through an abdominal wall of a patient when forming a stoma, and an inflatable member 502 configured to be positioned proximate the stoma to open and close the stoma, as desired. In this embodiment, however, the inflatable member 502 is housed within the base portion 538 so that the inflatable member 502 is received within the patient's body to form an artificial sphincter of the stoma. In other words, as will be described in further detail below, the inflatable member 502, in this embodiment, does not extend about an exterior wall of the target organ, but is received within and/or in an anterior to an end 50 of a target organ (e.g., colon).

The base portion 538 may be substantially similar to the base portion 438 of the system 400, including a tubular body 542 extending from a first end 544 to a second end 546. The base portion 538, however, may be formed of a soft polymer that is readily sutured to otherwise adhered to skin 56 and the tissue of the target organ to form a bridge between the end 50 of the target organ and the skin 56 surrounding the opening of the abdominal wall. In an exemplary embodiment, the base portion 538 is formed of a biocompatible mesh or bio-adhesive that enables ingrowth and integration of tissue.

Similarly to the base portion 438, in an exemplary embodiment, the base portion 538 includes a flange 550 at the second end of the tubular body 542. The flange 550 is configured to be attached to and/or sutured to the skin 56 upon insertion of the ostomy device 501 within the opening of the abdominal wall. In an exemplary embodiment, the end 50 of the target organ may be attached to a portion of the tubular body 542 so that attachment or suturing of the base portion 538 to the skin 56 forms the stoma. In another embodiment, a portion of the target organ is drawn through the tubular body 542 so that edges of the wall of the target organ may be sutured or otherwise attached to the skin 56 along with the flange 550.

As discussed above, the inflatable member 502 is received within the tubular body 542 and is movable between the deflated configuration (shown in FIG. 20), in which the stoma is open, and an inflated configuration (shown in FIG. 21), in which the inflatable member 502 is inflated, causing it to expand and thereby close the stoma. Similarly to the inflatable members 102-402 described above, the inflatable member 502 of this embodiment is substantially toroidal defining a hole 506 therethrough so that, when the inflatable member 502 is in the deflated configuration, stool is permitted to pass from the target organ, through the hole 506 and the stoma, and out of the body. In the inflated configuration, the inflatable member 502 expands, reducing a size of the hole 506 preventing stool and other waste from passing therethrough. In an exemplary embodiment, outermost edges of the inflatable member 502 are fixed to an interior 543 of the tubular body 542 so that the inflatable member 502 maintains its desired position proximate the stoma upon implantation into the body.

Similarly to the systems described above, the inflatable member 502 may be moved between the deflated and inflated configurations via a tubing 504 extending therefrom, to an exterior of the patient body. The tubing 504 extends from a first end 508 connected to the inflatable member 502 to a second end 510 that extends out of the patient's body to a location accessible to the patient. The second end 510 of this embodiment includes a control valve 516 controlling flow to and/from an inflation device (e.g., syringe) configured to provide inflation fluid (e.g., gas or liquid) to the inflatable member 502, as desired.

In an exemplary embodiment, the ostomy device 501 further comprises a cylindrical disk 536 extending about a portion of the base portion 538, within which the inflatable member 502 is received, to provide a radially inward pressure to the inflatable member 502 so that, as the inflatable member 502 is moved from the deflated configuration to the inflated configuration, the inflatable member 502 expands radially inward such that the hole 506 extending therethrough decreases in size to move the stoma to the closed position.

Although the exemplary embodiments show and describe the inflatable members 102-502 as being generally toroidal, it will be understood by those of skill in the art that the inflatable members 102, 502 may take any of a variety of shapes and/or configurations so long as movement between the deflated and inflated configurations opens and closes the stoma. For example, in an exemplary embodiment, as shown in FIGS. 22-23, an ostomy device 601 may be substantially similar to the ostomy device 501 described above, comprising an inflatable member 602 received within a base portion 638. Rather than having a toroid shape, however, the inflatable member 602 may be comprised of a plurality of lobes 603, each of which is attached to an interior of the 643 of the base portion 538 and movable between a deflated configuration, in which a hole 606 is defined between the lobes 603 to open a stoma, and an inflated configuration, in which the lobes 603 are inflated to expand, thereby reducing a size of the hole 606 to close the stoma.

As shown in FIG. 24, a system 700 according to another exemplary embodiment is substantially similar to the system 500 described above except as noted below. The system 700 comprises an ostomy device 701 including an inflatable member 702 configured to open and close a stoma 78, as desired. Upon formation of the stoma, however, the ostomy device 701 is configured to be inserted through the stoma 78 and into an interior portion of a target organ 70 (e.g., colon) so that the inflatable member 702 is positioned adjacent an abdominal wall 72 through which the stoma 78 is formed, and a tube element 782 extending from a through-hole 706 (e.g., a through-channel) of the inflatable member 702 extends out of the stoma 78 to an exterior of the body. Thus, when the stoma 78 is open, stool may be pass from the target organ 70, through through-hole 706 and the tube element 782, and out of the body.

In an exemplary embodiment, the inflatable member 702 may be substantially similar to the inflatable members 102-602 described above, the inflatable member 702 being movable between a deflated configuration and an inflated configuration. In the deflated configuration, the stoma is in an open position so that stool may pass from the target organ through the through-hole 706 and out of the stoma to be drained directly into, for example, a toilet, or collected in a bag or other container. In the inflated configuration, inflation fluid (e.g., gas or liquid) is received within the inflatable member 702 so that the inflatable member expands to reduce a size of the through-hole 706 toward a closed position of the stoma. Thus, in this inflated configuration, stool is prevented from passing through the through-hole 706.

In exemplary embodiment, the through-hole 706 tapers from a first end 744 toward a second end 746 so that stool is directed toward the tube element 782 extending from the second end 746. The tube element 782 extends from the second end 746 of the through-hole 706 of the inflatable member 702 to an outlet end 784 which, in an operative configuration in which the inflatable member is implanted within the colon, as described above, extends outside of the body.

According to a further exemplary embodiment, as shown in FIGS. 25-26, the ostomy device 701 further includes a substantially planar faceplate 754 extending about the outlet end 784 of the tube element 782 so that a through hole 755 of the faceplate 754 is aligned with the outlet end 784 of the tube element 782. A spacer 752 extends from a surface 756 of the faceplate 754, which faces the stoma 78, so that, in the operative configuration, the spacer 752 abuts exterior skin 76 of the abdominal wall 72 and extends between the skin 76 and the faceplate 754 to distance the faceplate 754 from the skin 76. In an exemplary embodiment, the spacer 752 is expandable along a length the tube element 782 so that the distance of the faceplate 754 from the skin may be adjusted, as desired. A length of the spacer 752 may be increased by supplying the inflation fluid thereto, and reduced by releasing the inflation fluid therefrom. It will be understood by those of skill in the art that the spacer 752 also aids in maintaining the inflatable member 702 in a desired position along the target organ, preventing migration of the inflatable member 702 therealong.

In an exemplary embodiment, the ostomy device 701 includes a secondary drainage control element 786 which extends into the tube element 782, across a channel thereof, to provide additional leakage protection. The secondary control element 786 is movable between a closed configuration, in which the secondary control element 786 extend across and blocks the channel of the tube element 782 to prevent waste within the tube element 782 from leaking therefrom, and an open configuration, in which the secondary control element 786 unblocks the channel of the tube element 782 so that the stool that has been received within the channel of the tube element 782 is permitted to be drained therethrough, toward the outlet end 784 thereof and out of the body. The secondary control element 786 may be particularly useful for situations in which stool has exited the stoma, but remains within the tube element 782. The secondary control element 786 prevents the stool from leaking out of the tube element 782.

It will be understood by those of skill in the art that the secondary control element 786 may have any of a variety of configurations so long as the secondary control element 786 is movable between the open and closed configurations, as discussed above. In one example, the secondary control element 786 may be a balloon that expands to fill the channel of the tube clement 782 as it is moved from the open configuration toward the closed configuration. In another example, the secondary control element 786 may be configured as a flap movably connected to an interior of the tube element 782.

In an exemplary embodiment, the ostomy device 701 also includes an irrigation channel 788 extending along an interior of the tube element 782 and into the through-hole 706 of the inflatable member 702 to provide irrigation to the through-hole 706 and the portion of the colon in which the inflatable member 702 is received.

In this embodiment, the faceplate 754 includes a plurality of ports 790 each of which is configured to be connected to one or more of a plurality of fluid (e.g., air or water) sources and/or pump devices, actuators, or other control means for controlling, for example, movement of the inflatable member 702 between the deflated and inflated configurations, movement of the secondary control element 786 between the open and closed configurations, adjustment of the spacer 752, and irrigation via the irrigation channel 788. In an exemplary embodiment, the fluid sources/pump devices may include a syringe or a hand pump. For example, a first port 790A may be in communication with the inflatable member 702 and connectable to, for example, a syringe or hand pump, so that the inflatable member 702 may be moved between the deflated and inflated configurations to open and close the stoma, respectively, as desired.

A second port 790B is in communication with the secondary control element 786 to move the secondary control element 786 between the open and the closed configurations, as described above. For example, where the secondary control element 786 is configured as a balloon, the second port 790B may also be connectable to a pump device such as a syringe. A third port 790C may be in communication with the spacer 752 and connectable to a pump device for lengthening and shortening the spacer 752 along the tube element 782, as desired.

A fourth port 790D is in communication with the irrigation channel 788 and connectable to a fluid source. It will be understood by those of skill in the art that the although each of the ports 790A-790D is described as connectable to separate pump and/or fluid (i.e., gas/liquid) sources, the ports 790A-790D may be connected to one or more pumps and/or fluid (i.e., gas/liquid) sources, each of the ports 790A-790D may include a valve which may be opened and closed, as desired, to permit gas or liquid flow therethrough.

In another exemplary embodiment, as shown in FIG. 27, rather than or in addition to the secondary control element 786, leakage of residual waste remaining in the tube element 782 is prevented by a clamp 787, which may be clamped over the tube element 782 after drainage thereof. The tube element 782, in this embodiment, is configured so that the outlet end 784 extends beyond the faceplate 754 to facilitate clamping thereof. In an alternative embodiment, as shown in FIG. 28, rather than having a tube extending from the through-hole 706 of the inflatable member 702, a tube or an ostomy bag 714 may be directly coupled to the faceplate 754. Although not shown, similarly to the stepped groove described with respect to the head portion of the system 400, the faceplate 754 may include a stepped groove within which a portion of the bag 714 may be received to engage the bag 714 during a stool collection.

According to another exemplary embodiment, as shown in FIG. 29, a system 800 may be substantially similar to the system 700 described above, comprising an inflatable member 802 configured to be inserted into a stoma. The inflatable member 802, however, does not include a through-hole, but rather, is configured as an extraction balloon. In an operative configuration, the inflatable member 802 is configured to be inserted into the stoma in a deflated configuration. Upon insertion through the stoma and into a colon, the inflatable member 802 may then be moved to an inflated configuration, in which the inflatable member 802 expands so that contents of the colon may be swept thereout via the expanded inflatable member 802. In other words, the expanded inflatable member 802 is drawn along the colon and out of the stoma to sweep the waste therefrom.

The system 800 further comprises a shaft or tubing 804 extending from a first end 808 connected to the inflatable member 802 to a second end 810 which, in an operative position, extends out of the stoma. The tubing 804 should have sufficient rigidity so that the inflatable member 802 may be inserted through the stoma and guided into the intestine via the tubing 804. In an exemplary embodiment, the system 800 further comprises a stop 850 proximate the second end 810 of the tubing 804. The stop 850 may be formed of a rigid or semi-rigid material extending about a portion of the tubing 804, the stop 850 sized and shaped so that, when the inflatable member 802 is inserted through the stoma and into the target organ to a threshold distance, the stop 850 abuts the skin of the abdominal wall to prevent the inflatable member 802 from being moved any further into the target organ. The stop 850, therefore, prevents migration or loss of the inflatable member 802 during use of the system 800.

To move the inflatable member 802 between the deflated and inflated configurations, the system 800 includes a connector 811 (e.g., a luer connector) at the second end 810 of the tubing 804. The connector 811 is configured to connect the tubing 804 to, for example, a syringe 826 for inflating the inflatable member 802. In an alternative embodiment, the syringe 826 may be permanently connected to the tubing 804 at the second end 810 via a three-way valve. As will be understood by those of skill in the art, a plunger of the syringe 826 may be moved to supply air to the inflatable member 802 to expand/inflate the inflatable member 802 or to draw air out of the inflatable member 802 to deflate the inflatable member 802, as desired.

As shown in FIG. 30, a system 900 according to another exemplary embodiment may be substantially similar to the systems described above. The system 900, however, comprises a plurality of inflatable members 902A, 902B, 902C, 902D, each of which has a substantially toroidal shape so that the inflatable members 902A, 902B, 902C, 902B are positionable about varying portions of a target organ 90 (e.g., a colon) that is attached to an opening of an abdominal wall 92 to form a stoma 98.

In an exemplary embodiment, the inflatable member 902A may be substantially similar to the inflatable member 102, positioned along an exterior skin 96 of the abdominal wall, about the target organ 90, so that when edges of the target organ 90 are stitched to the skin 96, the inflatable member 902A is secured between the skin and the exterior of the target organ to form an artificial sphincter of the stoma 98. The inflatable member 902A may be moved between the deflated and inflated configurations to open and close the stoma, as desired. The remaining inflatable members 902B, 902C, 902D are positioned internally, proximate an inner surface 97 of the abdominal wall 92, about the target organ 90, along varying portions of a length thereof, to form an artificial rectum. In particular, when the inflatable member 902A is moved from the inflated configuration toward the deflated configuration to open the stoma 98, each of the inflatable members 902B, 902C, 902D may be sequentially moved from a deflated configuration toward an inflated configuration, starting with the inflatable member 902B farthest from the stoma 98, to apply a radially inward pressure on the target organ 90 to push waste toward the stoma 98 and out of the body.

Once the stool has been pushed out of the stoma 98, as desired, the inflatable members 902B, 902C, 902D may be moved once again toward the deflated configuration, and the stoma 78 may be closed be inflating the inflatable member 902A. Although the system 900 is shown and described as including the inflatable member 902A to form an artificial sphincter, it will be understood by those of skill in the art that system may be utilized without the inflatable member 902A to form the artificial sphincter and so that just the inflatable members 902B, 902C, 902D are used to form the artificial rectum.

As described above, the inflatable member 902A may be moved between the deflated and inflated configuration substantially similarly to the inflatable member 102. In particular, the system 900 may include a tubing (not shown) extending from the inflatable member 902A via which an inflation fluid (e.g., gas or liquid) may be supplied to and from the inflatable member 902A. Similarly, the system 900 also includes a tubing 990 for suppling an inflation fluid (e.g., gas or liquid) via, for example, a syringe coupled thereto, to the inflatable members 902B, 902C, 902D. In an exemplary embodiment, the tubing 990 may be connected to and in communication with the inflatable member 902B which, in the operative configuration, is farthest from the stoma 98.

The inflatable members 902B, 902C, 902D may be connected to an in communication with one another via a narrow channel 992 so that upon inflation of the farthest one of the inflatable members 902B, the inflation fluid provided via the tubing 990, flows through the narrow channel 992 into a next farthest inflatable member 902C, and upon inflation of the next farthest inflatable member 902C, the inflation fluid flows into a final one of the inflatable members 902D so that the inflatable members 902B, 902C, 902D are sequentially inflated, pushing the waste through the target organ 90 toward the stoma 98. In a further embodiment, there may be a valve in between inflatable members requiring a certain pressure before fluid may pass through it. For example, there may be a valve in between the inflatable member 902B and the next farthest inflatable member 902C in the channel 992, so that the inflatable member 902B is fully inflated before the next farthest inflatable member 902C is inflated. Although the system 900 shows and describes three of the inflatable members 902B, 902C, 902D forming the artificial rectum, it will be understood by those of skill in the art that the artificial rectum may be formed via any number of inflatable members positioned internally, along the target organ 90.

It will be apparent to those skilled in the art that various modifications may be made in the present disclosure, without departing from the scope of the disclosure. Furthermore, those skilled in the art will understand that the features of any of the various embodiments may be combined in any manner that is not inconsistent with the description and/or the functionality of the embodiments.

1-15. (canceled)

16. An ostomy device, comprising:
an inflatable member defining a hole extending therethrough, the inflatable member configured to extend about a portion of an intestine proximate a stoma, in an operative configuration, the inflatable member movable between a deflated configuration, in which the stoma is maintained in an open position, and an inflated configuration, in which the inflatable member expands to decrease a size of the hole such and close the stoma; and
a tubing extending from a first end connected to the inflatable member to a second end, a channel of the tubing in communication with an interior of the inflatable member such that inflation fluid is configured to flow through the tubing to move the inflatable member between the deflated and inflated configurations.

17. The device of claim 16, wherein the hole of the inflatable member is configured to receive the portion of the intestine proximate the stoma therein so that, in the inflated configuration, the inflatable member expands to apply a radially inward pressure about the portion of the inflatable member received therein to close the stoma.

18. The device of claim 16, further comprising:
a bag configured to be attached to an abdominal wall about the stoma such that the tubing extends into an interior thereof.

19. The device of claim 18, further comprising:
a valve including a first port extending into the interior of the bag to be coupled to the second end of the tubing and a second port extending along an exterior of the bag to be coupled to an inflation device for providing inflation fluid to the inflatable member, the valve movable between an open configuration, in which a pathway between the first and second ports is open to allow a flow therebetween and inflate the inflatable member, and a closed configuration, in which the pathway between the first and second ports is closed.

20. The device of claim 18, wherein the second end of the tubing includes a luer connector which extends to an exterior of the bag and is configured to be connected to an inflation device.

21. The device of claim 20, further comprising:
a clamp configured to be clamped over the bag and the tubing to maintain the inflatable member toward the inflated configuration.

22. The device of claim 17, wherein the inflatable member forms a partial ring, extending from a first end to a second end.

23. The device of claim 16, wherein the inflatable member is configured be inserted through the stoma and received within the portion of the intestine proximate the stoma so that the hole of the inflatable member is aligned with a pathway through the intestine, the device further comprising a tube element extending from a connecting end connected to the hole of the inflatable member to an outlet end to direct intestinal contents passing through the hole of the inflatable member out of the stoma via the outlet end.

24. The device of claim 23, further comprising:
a secondary control element housed within the tube element and movable between a closed configuration, in which the secondary control element extends across a channel of the tube element to block flow of intestinal contents therethrough, and an open configuration, in which the secondary control element unlocks the channel of the tube element to permit a passage of intestinal contents therethrough.

25. The device of claim 24, wherein the secondary control element is configured as a balloon.

26. The device of claim 24, further comprising:
a faceplate coupled to the outlet end of the tube element; and
an expandable spacer configured to extend between the faceplate and an exterior surface of an abdominal wall, in the operative configuration, a length of the spacer configured to be adjustable along a length of the tube element to attain a desired distance between the faceplate and the exterior surface of the abdominal wall.

27. The device of claim 26, wherein the faceplate includes a plurality of ports, each of which is configured to be connected to one or more of a plurality of fluid sources for controlling an inflation of the inflatable member, a movement of the secondary control element between the open and closed configurations, and adjustment of the length of the expandable spacer.

28. A system for draining intestinal contents via a stoma, comprising:
a plurality of inflatable members, each of which is configured to be positioned about a portion of an intestine, proximate an interior surface of an abdominal wall within which the stoma is formed, the inflatable members configured to be moved from a deflated configuration toward an inflated configuration, in which the inflatable members are expanded radially inward to squeeze a portion of the intestine received therein, the plurality of inflatable members configured to be moved from the deflated configuration toward the inflated configuration sequentially, from an inflatable member farthest from the stoma to an inflatable member closest to the stoma, so that intestinal contents are pushed through the intestine toward the stoma.

29. The system of claim 28, further comprising:
a tubing extending from a first end connected to the inflatable member farthest from the stoma to a second end, a channel of the tubing in communication with an interior of the inflatable member farthest from the stoma such that inflation fluid is configured to flow through the tubing to the inflatable member farthest from the stoma; and
a channel connecting the plurality of inflatable members so that upon inflation of the inflatable member farthest from the stoma, inflation fluid is permitted to flow into a next adjacent inflatable member to sequentially inflate the plurality of inflatable members from the inflatable member farthest from the stoma to the inflatable member closest to the stoma.

30. The system of claim 28, further comprising:
an artificial sphincter having a substantially toroid shape to define a hole extending therethrough, the hole configured to receive a portion of the intestine proximate the stoma therethrough, in an operative configuration, the inflatable member movable between the deflated configuration, in which the stoma is maintained in an open position, and the inflated configuration, in which the inflatable member expands to decrease a size of the hole such that a radial inward pressure is applied to a portion of the intestine received therein to close the stoma.

31. A method for performing an ostomy procedure, comprising:
making an incision through an abdominal wall and drawing an end of a colon through the incision;
drawing an end of the colon through the incision and positioning an inflatable member along an exterior surface of the abdominal wall and about a portion of the colon;
stitching edges of the colon to the exterior surface of the abdominal wall to form a stoma such that the inflatable member is secured to between the exterior surface of the abdominal wall and the end of the colon; and
moving the inflatable member between a deflated configuration and an inflated configuration to open and close the stoma, respectively, as desired.

32. The method of claim 31, wherein moving the inflatable member between the deflated and inflated configurations includes providing inflation fluid to and from the inflatable member via a tubing extending therefrom.

33. The method of claim 31, further comprising:
attaching an ostomy bag to the exterior surface of the abdominal wall such that intestinal contents flow from the stoma into the ostomy bag when the stoma is open.

34. The method of claim 33, wherein attaching the ostomy bag includes connecting a first port of a valve of the ostomy bag to the inflatable member and a second port of the valve to an inflation device for providing inflation fluid to the inflatable member, the valve movable between an open configuration, in which a flow between the inflatable member and the inflation device is permitted, and a closed configuration, in which flow between the inflatable member and the inflation device is prevented.

35. The method of claim 31, wherein the inflatable member is positioned along the exterior surface of a skin by implanting a base portion into an opening of the abdominal wall formed via the incision, the inflatable member housed within a head portion that is releasably coupled to the base portion.
